(19) United States
(12) Patent Application Publication
(10) Pub. No.: US 2024/0123012 A1
(43) Pub. Date: Apr. 18, 2024

(54) THERAPEUTIC COMPOSITION BASED ON DHODH-INHIBITING NEUROLAENA LEAVES FOR THE TREATMENT OF RNA VIRUS INFECTIONS

(71) Applicant: PHYTOBOKAZ, Gourbeyre (FR)

(72) Inventor: Henry JOSEPH, Gourbeyre (FR)

(21) Appl. No.: 18/264,918

(22) PCT Filed: Feb. 9, 2022
(86) PCT No.: PCT/EP22/53144

§ 371 Date: Aug. 9, 2023

(30) Foreign Application Priority Data
Feb. 10, 2021 (FR) FR2101262
Jan. 5, 2022 (FR) FR2200066

(51) Int. Cl. A61K 36/28; A61P 31/14

(52) U.S. Cl. CPC A61K 36/28; A61P 31/14; A61K 2236/333; A61K 2236/51

(57) ABSTRACT

A therapeutic composition includes a mother tincture of a leaf extract of a plant of the genus “Neurolaena” and the species “lobata” for use as a drug inhibiting human dihydroorotate dehydrogenase (DHODH).

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### The Names of Parties to a Joint Research Agreement

Not applicable.

### Incorporation-by-Reference of Material Submitted on a Compact Disc or as a Text File Via the Office Electronic Filing System (Efs-Web)

Not applicable.

### Statement Regarding Prior Disclosures by the Inventor or a Joint Inventor

Not applicable.

### BACKGROUND OF THE INVENTION

### 1. Field of the Invention

The present invention relates to a therapeutic composition comprising a mother tincture of a leaf extract of a plant of the genus “Neurolaena” and the species “lobata” for use as a drug inhibiting human dihydroorotate dehydrogenase (DHODH.

### 2. Description of Related Art Including Information Disclosed Under 37 CFR 1.97 and 37 CFR 1.98

RNA genome viruses are responsible for numerous human pathologies such as, for example, influenza, dengue, hepatitis C, measles, infant bronchitis, or more recently, the Covid-19 coronavirus.

The conventional therapeutic arsenal against RNA genome viruses consists of targeting the activity of an essential viral protein in the virus cycle. Such essential proteins may in particular consist of RNA polymerase, integrase, a helicase or a protease. However, although these viral proteins can be preserved in several RNA genome viruses, the development of broad-spectrum therapeutic molecules, active against different viruses, remains relatively limited.

In addition, the plasticity of viral genomes, as well as the ability of RNA genome viruses to adapt and change, facilitates the rapid appearance of escape mutants. These mutants make treatment with broad-spectrum therapeutic molecules, which target a viral protein essential to the cycle of the virus, ineffective.

In order to overcome these problems of the therapeutic targets mutating, a new therapeutic approach targeting the host cell, rather than the virus itself, has been developed in recent years. The principle of this new approach is to block the cell mechanisms essential to viral replication, so as to prevent viral proliferation by stimulating the innate immune response in cells infected by an RNA genome virus.

One of the known cell mechanisms, essential to viral replication, is the pyrimidine biosynthesis cycle. Indeed, pyrimidine is crucial for the survival of human cells, in particular when the latter are pathogen's host cells, in particular RNA genome viruses.

More particularly, in mammals and in human cells, the synthesis of the pyrimidine is carried out by two biosynthses pathways: the “de novo pyrimidine pathway”, and another “salvage pathway” which is carried out under particular physiological conditions. Most human parasites do not have the “salvage pathway” for synthesizing pyrimidines. However, the pyrimidines are necessary for the manufacture of pyrimidine nucleotides. These pyrimidine nucleotides are essential to cell survival and to cell multiplication. Thus, the blocking of the “de novo pyrimidine pathway” is considered to be a therapeutic means effective for selectively targeting human parasites without affecting the human host and its normal cellular functioning.

In other words, all human pathogens, including RNA genome viruses, are deficient in the “salvage pathway” for pyrimidine biosynthesis. Therefore, the “de novo pyrimidine pathway”, of the host cell, is a targeted pathway in therapy to eliminate these pathogens, in particular to prevent the replication of the RNA viruses in the host cells.

The de novo pyrimidine biosynthesis pathway is a synthesis pathway which is carried out in several successive steps. This pathway and its various steps are shown in FIG. 1 of the attached drawings.

More particularly, the fourth step of the de novo pyrimidine biosynthesis pathway consists of a dehydrogenation of the dihydroorotate called “DHO” which leads to the formation of orotate “ORO”.

The enzyme, of the oxidoreductase type, which catalyzes the dehydrogenation reaction of the dihydroorotate, is dihydroorotate dehydrogenase, also known as “DHODH”.

During the dehydrogenation of DHO into ORO, there is an electron transfer between two cofactors, one of which is an electron donor and the other is an electron acceptor. For example, the electron transfer, during this dehydrogenation reaction of the dihydroorotate, is done by virtue of the flavin mononucleotide redox couple FMN/FMNH2 with the ubiquinone couple QH2/Q or with the nicotinamide adenine couple NAD+/NADH. DHODH binds to its FMN cofactor in conjunction with ubiquinone to catalyze the oxidation of dihydroorotate into orotate.

The de novo biosynthesis pathway of the pyrimidines ensures the synthesis of uridine 5-monophosphate, also called UMP. UMP serves as a precursor for the other pyrimidine nucleotides. These nucleotides are necessary and essential to cell division and to the metabolic activity of the host cell which is infected by the RNA genome virus. Thus, it has been demonstrated that inhibiting the DHODH of the host cells causes a fall in the amount of pyrimidines in the infected cells, which amplifies the antiviral innate immune response.

In a known manner, the DHODH enzymes are separated into two groups, the DHODHs of class 1 and the DHODHs of class 2. These two classes of DHODH are established as a function of their sequence similarity, of their binding sites, of their cell location and of their preferred substrate.

DHODH class 1 is cytosolic enzymes present in pathogens of the protozoa type.

DHODH class 2 are monomeric protein-type enzymes binding to the internal membrane of the eukaryotic mitochondria.

In other words, in humans, DHODH belongs to class 2, it is a mitochondrial protein located on the external surface of the internal mitochondrial membrane. Human DHODH has two domains consisting of the alpha/beta barrel domain containing the active site and the alpha helix domain; the latter forms the opening of a tunnel leading to the active site.

To date, there are several known inhibitors of human DHODH, such as brequinar, teriflunomide or leflunomide.

For example, leflunomide is used to treat rheumatoid arthritis or multiple sclerosis. Immunosuppressive effects of leflunomide were attributed to the exhaustion of the addition of pyrimidine for T cells or to more complex interferon- or interleukin-mediated pathways. The inhibition of human DHODH, by leflunomide is due to the attachment of leflunomide on alpha helix domain which forms the opening of a tunnel leading to the active site of the DHODH. Although marketed as an active ingredient in medications, leflunomide leads to side effects in 1% to 10% of patients, in particular diarrhea, nausea, vomiting, aphthous, abdominal pain, inflammation of the colon, headaches, inflammation of the tendons, accentuating natural hair loss, eczema, dry skin, increase of transaminases, or lowering white blood cell counts.

The inhibition of human DHODH by brequinar is carried out by the same mechanism as leflunomide, taking into account the opening of the tunnel leading to the active site. Brequinar was used as an anti-cancer treatment in the late 1980s; nevertheless as it is responsible for multiple undesirable side effects, it has not been accepted as medicine. In addition, the brequinar is recognized as highly toxic to human cells. Consequently, the idea of using it in therapy to combat viral infections was very quickly abandoned by the medical establishment.

Thus, in the case of the development of a pathology linked to a viral infection by an RNA-genome virus, known inhibitors of DHODH seem unsuitable for treating the viral symptoms of a patient, in particular due to their side effects and cytotoxic effects on the cells, without distinguishing the type of cell.

This is the reason why, in the case of a viral infection, in particular by an RNA virus, it is advisable to find an alternative solution to known inhibitors of DHODH, such as brequinar, teriflunomide, or leflunomide. This alternative solution, in addition to preventing the de novo pyrimidine pathway by inhibition of the enzymatic activity of the DHODH, is desired to be non-cytotoxic for all human cells and with a minimum of side effects in the event it is taken as medication.

In addition, in the current context, most patients are attentive to the origin and design modes of medications. In general, patients wish to minimize the proportion of synthetic components in the medicament. These synthetic components may be responsible for side effects, just like the active ingredient. Consequently, in addition to inhibiting human DHODH, the objective of the present invention also consists of finding a DHODH inhibitor which to the extent possible is of natural origin, of simple design with a small carbon footprint, in order to limit the side effects while treating RNA virus infection and the associated symptoms.

### BRIEF SUMMARY OF THE INVENTION

The purpose of the present invention is to overcome the disadvantages of the prior art, by proposing a therapeutic composition comprising a mother tincture of a leaf extract of a plant belonging to the genus “Neurolaena” and in this case “lobata” for use as a drug inhibiting human dihydroorotate dehydrogenase (DHODH).

This therapeutic composition has the advantage of having, as active ingredient inhibiting the activity of the DHODH, said mother tincture of Neurolaena lobata. This mother tincture is advantageously easy and quick to prepare, inexpensively at industrial scale. In addition, said mother tincture of the therapeutic composition of the invention is advantageously of natural origin, that is to say non-synthetic, while being recognized in the literature as being non-cytotoxic in vivo.

Consequently, said therapeutic composition has little or no side effects linked to its active ingredient of natural and non-cytotoxic origin. Following ingestion by a patient, said therapeutic composition of the invention inhibits the activity of the DHODH without having a cytotoxic effect. The setting of said therapeutic composition, including said mother tincture, generates an increase in the innate immune response in patients whose immune system is weakened by pathogens or any other diseases.

In addition, according to other features of the invention, said therapeutic composition is for use as a medicament in the treatment of symptoms associated with a viral infection of RNA-genome virus.

Said therapeutic composition, comprising a mother tincture of Neurolaena lobata having the property of inhibiting the activity of the DHODH and preventing the de novo pyrimidine synthesis pathway, is suitable in the case of viral infection by an RNA virus, in particular positive RNA.

Indeed, RNA viruses are deficient in the “pyrimidine salvage pathway”. The RNA viruses for their cell infection mechanism must use the “de novo pathway” of the host cell to synthesize the pyrimidine which is necessary in the cellular replication mechanism, in other words necessary for viral multiplication. Thus, the inhibition of the de novo pathway of the host cell prevents cell replication, that is to say viral replication of the RNA viruses. This is why the use of said therapeutic composition of the invention as a medicament for treating the symptoms associated with a viral infection of the RNA-genome virus is appropriate.

According to a preferred embodiment, the therapeutic composition of the invention is used as a drug in the treatment of symptoms associated with a viral infection of RNA-genome virus selected from the virus families of the following list: Coronaviridae, Flaviviridae, Orthomyxoviridae, or Togaviridae.

Most preferentially, the present invention also relates to a method for preparing a dry extract of a diluted mother tincture of dried leaves of Neurolaena lobata, characterized in that it comprises the following steps:
i) a mixture of dried leaves of Neurolaena lobata in sugar cane alcohol at 50° is prepared, said mixture having a mass concentration of between 15 and 20 g/L;
ii) said mixture is allowed to macerate under agitation for about 21 days;
iii) After maceration, the mixture is filtered on “cartridges” of porosity 50-75 μm and a liquid filtrate is obtained, corresponding to a mother tincture of dried leaves of Neurolaena lobata, and a solid retentate;
iv) said liquid mother tincture is diluted to ¼ by adding an aqueous solution, and an aqueous-alcoholic solution is thus obtained;
v) an evaporation of the alcohol contained in this solution by a rotary evaporator, until an aqueous solution is obtained;
vi) the aqueous solution thus obtained is consolidated and then freeze-dried to obtain a dry extract of the diluted mother tincture of dried leaves of Neurolaena lobata.

Advantageously, the mixture prepared in step i) has a mass concentration of between 16 and 17 g of dried leaves of Neurolaena lobata per liter (L) of sugar cane alcohol at 50°, and preferably equal to 16 g/L.

Most preferentially, during step iv), said mother tincture is diluted by mixing a volume equal to 0.75 L of filtrate and a volume equal to 2.25 L of water.

The invention also relates to a method for preparing a liquid solution from a dry extract of freeze-dried diluted mother tincture of dried leaves of Neurolaena lobata, said dry extract being obtained according to the method described above, said liquid solution being obtained by diluting said dry extract in water or in a pharmaceutically acceptable aqueous solvent, said liquid solution having a concentration of between 6,500 and 20,000 ng of dry extract per mL of aqueous solvent, preferably between 6,667 and 20,000 ng/mL.

In the present application, a pharmaceutically acceptable solvent is understood to mean a solvent that can be used in the preparation of a pharmaceutical composition and which has the characteristics of being non-toxic and biologically acceptable for veterinary use as well as human pharmaceutical use.

The present application also relates to a diluted mother tincture extract of dried leaves of Neurolaena lobata, in particular diluted to ¼ of the mother tincture, and freeze-dried, said extract being in a solution and having a final concentration of between 6,500 and 20,000 ng/mL (mass of freeze-dried dry extract of diluted mother tincture/volume of aqueous solution), for its use in the treatment of a viral infection due to the SARS-CoV-2 virus responsible for Covid-19.

Said dried leaves mother tincture extract of Neurolaena lobata diluted as described above can be obtained according to the method described above.

The mother tincture extract of Neurolaena lobata leaves is particularly indicated for use as a drug in the decrease in the production of cytokines, in particular IL-6 and IP-10, in the treatment of the serious forms of a viral infection due to the SARS-CoV-2 virus responsible for Covid-19.

### BRIEF DESCRIPTION OF THE SEVERAL VIEWS OF THE DRAWINGS

Other features and advantages of the invention will become apparent from the following detailed description of non-limiting embodiments of the invention, with reference to the attached figures.

FIG. 1 schematically shows the steps of the synthesis pathway of de novo pyrimidine in human cells, the fourth step of which consists of the dehydrogenation reaction, that is to say the action of the dihydroorotate dehydrogenase enzyme “DHODH” to transform the dihydroorotate “DHO” into orotate ORO.

FIG. 2 schematically represents the action inhibiting said composition of the invention on the mitochondrial human DHODH, within a human host cell infected by an RNA genome virus.

FIG. 3 shows a graph illustration of a trend curve, as a function of the contact time between the DHODH and its substrate, of the optical density measured at 600 nm of a sample at a concentration of 0.01 μg/mL consisting either of brequinar, or of a mother tincture of a leaf extract of Neurolaena lobata, named “H1”, or of another mother tincture of a leaf extract of Neurolaena lobata called “H2”.

FIG. 4 shows a graph illustration of an evolution curve, as a function of the contact time between the DHODH and its substrate, of the optical density measured at 600 nm of a sample at a concentration of 0.1 μg/mL consisting either of brequinar, or of “H1”, or of “H2”.

FIG. 5 shows a graph illustration of a evolution curve, as a function of the contact time between the DHODH and its substrate, of the optical density measured at 600 nm of a sample at a concentration of 1 μg/mL consisting either of brequinar, or of “H1”, or of “H2”.

FIG. 6 shows a graph illustration of the curve of the evolution of the percentage inhibition of the activity of the DHODH on its substrate, observed between 0 and 275 seconds of being in the presence of the sample, as a function of the concentration of the sample H1 or H2.

FIG. 7 shows a graph illustration of the curve of the evolution of the percentage inhibition of the activity of the DHODH on its substrate, observed between 0 and 275 seconds of being in the presence of the sample, as a function of the concentration of the sample H1, H2 or brequinar.

### DETAILED DESCRIPTION OF THE INVENTION

The present invention relates to a therapeutic composition comprising a mother tincture of a leaf extract of a plant belonging to the genus “Neurolaena” and in this case “lobata” for use as a drug inhibiting human dihydroorotate dehydrogenase (DHODH).

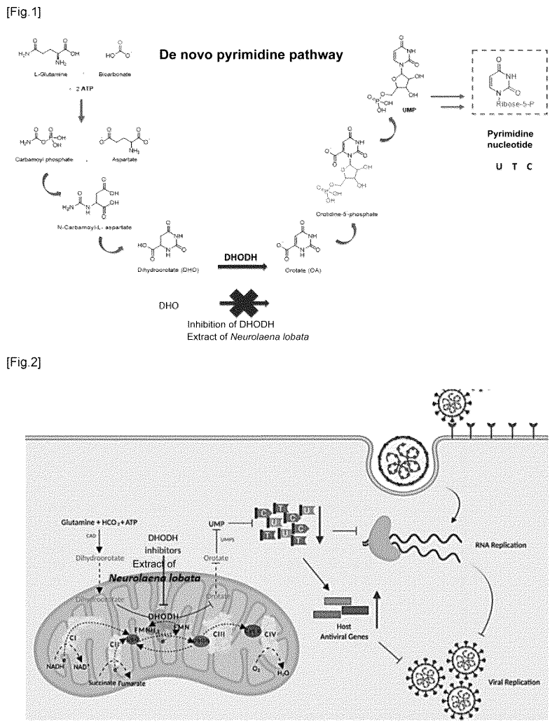

FIG. 1 schematically shows the de novo pathway for synthesizing pyrimidines comprising several steps, wherein step 4 involves the action of the human dihydroorotate dehydrogenase “DHODH”.

In the de novo pathway for synthesizing pyrimidines, the precursors of the pyrimidine nucleus are glutamine, aspartic acid and CO2. As can be seen in FIG. 1, in the first step, carbamoylphosphate is formed by carbamoylphosphate synthetase. In the second step, aspartate transcarbamylase catalyzes the formation of carbamoylaspartate. The latter is transformed in the third step, by dihydroorotase, into dihydroorotate “DHO”. Then, in the fourth step, the dihydroorotate dehydrogenase “DHODH” will catalyze the conversion of the “DHO” into orotate “ORO” which will be used as a reaction precursor to obtain uridine monophosphate “UMP”. UMP is useful in the polymerization mechanism with the RNA necessary for cell multiplication.

According to the invention, the mother tincture of a leaf extract of a plant belonging to the genus “Neurolaena” and in this case “lobata” is advantageously of natural origin. Indeed, Neurolaena lobata is a plant belonging to the family of Asteraceae that is found in the Antilles and in Central America, in particular in Guadeloupe. In those lands, this plant is easily cultivated and harvested because the agro-ecological and pedo-climatic conditions are favorable.

Thus, by its natural origin, the use of an extract of Neurolaena lobata advantageously makes it possible to reduce the risk of side effects during its use within a therapeutic composition to treat a disease.

In addition, to reinforce this idea, several scientific studies and publications have demonstrated that an extract of Neurolaena lobata does not have any toxicity in vivo (Gracioso J. S. et al., J. Pharm. Pharmacol. 1998, 50: 1425-1429; Gracioso J. S. et al., Phytomedecine, 2000, Vol. 7(4), pp. 283-289). In particular, in mice, after ingesting, by oral pathway, a dose of 5000 mg/kg of hydro-alcoholic extract of the shoots of Neurolaena lobata, no physiological toxicity was observed after several days.

As a result, in addition to its natural origin, the choice of an extract of Neurolaena lobata, as an active ingredient of a therapeutic composition aiming to inhibit the activity of DHODH, advantageously meets the objective of the invention to be non-cytotoxic for all human cells, in particular those involved in the immune response, at the concentrations that have been chosen.

According to the invention, said mother tincture of a leaf extract of Neurolaena lobata consists of a hydroalcoholic solution.

According to a preferred embodiment, the mother tincture is produced from an extract only of leaves of a Neurolaena lobata plant.

According to the invention, said therapeutic composition is in a dosage form for oral ingestion. For example, said therapeutic composition is in liquid form, for example in the form of a syrup, or in solid form, for example in the form of a tablet.

According to the invention, said therapeutic composition comprises as active ingredient said mother tincture of Neurolaena lobata having an inhibitory effect on the activity of the DHODH, as well as other excipients allowing its dosage formulation. The interactions that can exist between these excipients and said mother tincture do not influence and do not impact the inhibitory effect of the DHODH.

Preferably, said therapeutic composition of the invention comprises only excipients of natural origin, which generate few or no side effects when they are in a formulation with said mother tincture of Neurolaena lobate.

According to a preferred embodiment, said therapeutic composition is used as a drug in the treatment of symptoms associated with a viral infection.

More specifically, said therapeutic composition of the invention is used as a drug in the treatment of symptoms associated with an infection with an RNA-genome virus selected from the families of viruses of the following list: Coronaviridae, Flaviviridae, or Togaviridae:
among Coronaviridae, SARS-CoV-2, responsible for Covid-19, will be mentioned;
among Flaviviridae, mention will be made of the genus Hepacivirus, the only representative of which is the virus responsible for hepatitis C, and, in the genus Flavivirus, the virus responsible for yellow fever or the Zika virus and,
among the Togaviridae, mention will be made of the virus responsible for chikungunya.

Indeed, for their viral replication within the host cell, the RNA viruses require that the de novo pathway for synthesizing pyrimidines of the host cell be functional. These viruses lack a pyrimidine synthesis pathway. However, if this cellular pathway is blocked by the use of the therapeutic composition of the invention inhibiting the activity of DHODH, viral replication of the RNA viruses within the host cell will no longer be possible, as that cell lacks both the de novo pathway and the pyrimidine synthesis salvage pathway.

FIG. 2 shows the mechanism of action of the composition of the invention on the inhibition of DHODH in a human cell infected by an RNA-genome virus. Due to the mother tincture of a leaf extract of Neurolaena lobate of the composition of the invention, the dehydrogenation reaction carried out by DHODH in the mitochondria does not take place. The de novo pathway for the synthesis of the pyrimidine is blocked, and the salvage pathway cannot be used by the virus which lacks it. UMP and pyrimidine bases of nucleotides cannot be used by the RNA virus to start viral replication.

Consequently, no virion cannot be manufactured by the host cell, even after introduction of the viral RNA genome within said host cell. The inhibition of the DHODH prevents the viral multiplication of the RNA genome viruses and prevents the production and the output of virions outside the cell membrane of the host. Thus, the use of the composition of the invention, with an action inhibiting the activity of the DHODH prevents the viral multiplication and the development of the symptoms associated with the presence of the pathogen within the organism. The composition of the invention therefore constitutes a therapeutic means effective for treating viral infections, in particular by RNA genomes.

As a result, the use of the therapeutic composition of the invention as a drug in the treatment of symptoms associated with an infection with an RNA genome virus consists of a solution for combating infection and limiting viral multiplication.

The therapeutic composition of the invention is a good alternative to the existing solutions for treating viral disease targeting the inhibition of DHODH. This specific use makes it possible to prevent viral replication within the host cell, by inhibiting the de novo pyrimidine synthesis pathway, while increasing the immune response by the defense cells and without a cytotoxic effect.

The present therapeutic composition of the invention therefore consists of an alternative solution making it possible to inhibit DHODH, that is to say to block the de novo pathway of synthesis of pyrimidines necessary for the replication of the RNA viruses, without being invasive and destructive of the cells involved in the immune response against the pathogen.

The therapeutic composition of the invention also has the advantage of being easy to manufacture while being as natural as possible in the eyes of the consumers and patients who would be using it.

The results of the experiments below are intended to show the effect of inhibiting DHODH by the therapeutic composition of the invention.

Other results of experiments obtained in vitro detailed below show, in particular, the action of the therapeutic composition of the invention in particular on the serious forms caused by a viral infection with SARS-CoV-2 virus responsible for Covid-19.

Indeed, these results demonstrate a particularly interesting action of the therapeutic composition of the invention comprising a mother tincture of a leaf extract of a Neurolaena lobate plant on the decrease in the rate of certain cytokines released by the cells after infection by the SARS-CoV-2 virus.

The cytokines are proteins naturally synthesized by immune cells to mediate the immune response following infection by a pathogen. They promote a natural inflammatory reaction allowing the infected organism to defend against the pathogen.

However, in certain cases of SARS-CoV-2 infections, the release of cytokines, in particular in lung cells, is so important that it triggers a “cytokine storm”. This runaway of the immune system leads to a hyperinflammatory reaction liable to destroy tissues, to cause acute respiratory distress syndromes, which can lead to physiological deteriorations, or even to become lethal to the person in whom this reaction is triggered.

The results of tests, shown below, carried out in vitro on a therapeutic composition in accordance with the invention comprising a mother tincture of a leaf extract of a Neurolaena lobata plant demonstrate that cytokine production can be substantially decreased by the action of said composition.

To return now to the results of tests relating to the inhibition of the action of the DHODH, tests were conducted on a therapeutic composition of the invention comprising a mother tincture of a leaf extract of a Neurolaena lobata plant.

This mother tincture constitutes the sample to be tested. The measurement of the inhibition of the action of DHODH on its substrate is carried out in a conventional multi-well plate with a transparent wall. The wells contain the samples to be tested, the DHODH and its substrate.

To evaluate the inhibition of the DHODH by the sample, the parameter of the optical density called “OD” is used. Indeed, in each well, the “OD” is measured at a wavelength of 600 nm, at several time intervals, for a duration of 5 min.

More specifically, each well comprises the diluted or non-diluted sample, the enzyme of the DHODH and its colorimetric substrate diluted in a test buffer. Said colorimetric substrate comprises DHO which can be converted into ORO by action of the DHODH.

Over time, the consumption of the colorimetric substrate DHO by the enzyme DHODH results in a reduction in the OD. This decrease indicates the conversion of the colored DHO into uncolored ORO by the activity of the DHODH. In other words, the DHO is consumed, reduced to ORO by the activity of the DHODH, which causes a modification of the OD measured.

In the event of inhibition of the activity of the DHODH by the sample, the OD remains stable over time. Indeed, in the case of inhibition, the colorimetric DHO substrate will not be transformed by the DHODH into ORO, the OD therefore will remain that of the initial DHO.

To carry out the experiments, the following were used:
a solution of class 1 enzyme called “rhDHODH” for “human recombinant DHODH”,
a test buffer consisting of 50 mM Tris, 150 mM KCl and 0.1% Triton® X-100, at pH 8,
a substrate mixture of DHODH containing: 2 mM of L-dihydroorotic called “DHO”, 0.2 mM of decylubiquinone called “Q” and 0.12 mM of 2,6-Dichloroindophenol sodium salt hydrate called “DP IP” in the test buffer,
a mother tincture sample of a leaf extract of a Neurolaena lobata plant, diluted or not, in a dilution buffer consisting of dimethyl sulfoxide called “DMSO”.

It should be noted that in the mixture of substrate of DHODH, the L-dihydroorotic called “DHO” constitutes the colorimetric substrate used by DHODH during the dehydrogenation reaction.

In the substrate mixture, the decylubiquinone called “Q” and Dichloroindophenol sodium salt hydrate called “DPIP” are the electron acceptors and donors. The transfer of these electrons makes it possible to carry out the dehydrogenation reaction by the oxidoreductase DHODH.

Preparation of the mother tincture of a leaf extract of a Neurolaena lobata plant constituting sample H1:

The H1 mother tincture of a leaf extract of a Neurolaena lobata plant is obtained by performing the following method steps:
The leaves of Neurolaena lobata are harvested,
Said leaves are dried,
Said dried leaves are ground until a powder is obtained,
The powder is macerated in a sugar cane alcohol solution for 21 days until a macerate is obtained, the ratio of the amounts being 1 g of powder for every 62.5 mL of sugar cane alcohol solution,
Said macerate is filtered until a filtrate is obtained, called H1.

In the aforementioned method, according to a preferred embodiment, the leaves can be dried in a stream of hot air, at a temperature preferably below 40° C., for approximately 120 hours, until they have a residual moisture content of the order of 6 to 9, preferably 6.5 to 9%.

The moisture content is determined by any suitable methods known to the person skilled in the art. For example, the moisture content can be determined using a desiccator installed in a room having a temperature below 40° C., with a relative humidity ratio of less than 85% without direct exposure to the sun's rays, air current or vibrations.

For example, the desiccator with product code XM60 marketed by PRECISA MOLEN France, with a standard precision of 1 mg at high resolution and temperature ranges ranging from 30° C. to 230° C. with an increment of 1° C. can be used to measure the residual moisture content of the leaves.

Preferably, in the abovementioned protocol, the powder is macerated in a sugar cane alcohol solution at a temperature of 25° C. to 30° C., preferably 30° C., for approximately 21 days, with slow stirring every day for 12 h.

Preparation of the mother tincture of a leaf extract of a Neurolaena lobata plant constituting sample H2

The H2 mother tincture of a leaf extract of a Neurolaena lobata plant is obtained by performing the following method steps:
The leaves of Neurolaena lobata are harvested,
Said leaves are dried,
100 g of dried leaves are macerated in 1 L of a pure ethanol solution for 7 days until a macerate is obtained,
Said macerate is filtered on celite until a filtrate is obtained,
Said filtrate is concentrated by rotary evaporator.

Said concentrated filtrate is dried under pressure, in particular using a Schlenk line, until the sample H2 is obtained.

Protocol for analyzing the inhibition of DHODH by the composition of the invention.

In order to verify the inhibition of the DHODH by the therapeutic composition of the invention, two stock solutions, respectively called H1 and H2, were prepared.

The mother solution H1 is obtained by implementing the abovementioned method after harvesting the leaves of a Neurolaena lobate plant.

The H2 stock solution is obtained by implementing the aforementioned method.

In order to test the impact of the sample on the inhibition of the DHODH and to know “the dose-response effect”, each sample H1 and H2 was diluted in a DMSO buffer.

The dilutions of the H1 and H2 samples made it possible to obtain the following concentrations: in μg of sample/mL total solution in the well: 0.01 μg/mL; 0.1 μg/mL; 1 μg/mL; 10 μg/mL; 100 μg/mL; 1000 μg/mL indicated in table 1 and table 2 below.

To start the protocol, each dilution of the sample H1 or H2 was placed in the presence of the rh DHODH enzyme and the mixture of substrate within a well. In order to obtain an average of OD measured for a sample concentration H1 or H2, triplicates were carried out for each of the dilution concentrations of H1 and H2.

More specifically, to obtain the results below, the following steps of the protocol were carried out:
To each well comprising the dilutions of the H1 or H2 samples, the rh DHODH enzyme prepared on the same day is added in a test buffer solution,
The enzyme and the sample are left in each other's presence for 6 min at 37° C.,
50 μL of the aforementioned colored substrate mixture is then added, this consists of time 0,
It is measured in each well by immediate reading of the OD at 600 nm, at regular intervals, every 55 seconds, for 275 seconds.

The amount of enzyme added in each well is the same. In the protocol, the enzyme is added to the well so as to have a concentration of 0.06 μg of rh DHODH enzyme/mL of total solution in the well.

Table 1 below shows the results obtained for sample H1:

TABLE 1
Inhibition of the activity of the DHODH by sample H1

| | Concentration of the H1 samples in μg of | | | | | | |
|---|---|---|---|---|---|---|---|
| | H1/mL of total solution in the well | | | | | | Negative |
| Time | 0.01 | 0.1 | 1 | 10 | 1.00 | 1000 | control |
| (s) | μg/mL | μg/mL | μg/mL | μg/mL | μg/mL | μg/mL | E + S + T |
| 0 | 0.324 | 0.335 | 0.347 | 0.356 | 0.364 | 0.371 | 0.349 |
| 55 | 0.316 | 0.326 | 0.335 | 0.338 | 0.346 | 0.357 | 0.321 |
| 110 | 0.307 | 0.315 | 0.324 | 0.335 | 0.345 | 0.352 | 0.241 |
| 165 | 0.246 | 0.258 | 0.264 | 0.272 | 0.280 | 0.303 | 0.211 |
| 220 | 0.233 | 0.243 | 0.257 | 0.265 | 0.273 | 0.283 | 0.192 |
| 275 | 0.232 | 0.241 | 0.254 | 0.255 | 0.264 | 0.277 | 0.141 |
| Δ | 0.092 | 0.094 | 0.093 | 0.101 | 0.100 | 0.094 | 0.208 |
| % activated | 44.231 | 45.192 | 44.711 | 48.558 | 48.077 | 45.192 | 100.000 |
| % inhibition | 55.769 | 54.808 | 55.289 | 51342 | 51.923 | 54.808 | 0.000 |

In table 1, the first column gives the measurement-taking time intervals of the OD at 600 nm. In other words, this corresponds to the contact time of the sample H1 with the rh DHODH in the presence of the substrate mixture.

In table 1, the second line indicates the concentration of the sample H1 in the well. This concentration is expressed in μg of sample H1/mL of total solution in the well.

Each column of table 1 indicates the mean OD value measured at 600 nm of the triplicates for the same sample concentration H1, and for a defined time of contact with the rh DHODH and its substrate.

For example, as is visible in table 1, after 110 seconds contact between the sample H1 at a concentration of 0.1 μg/mL, the rh DHODH, and the mixture of colored substrate, the average value of the OD measured on the three wells, of identical capacity, is 0.315.

The row of the symbol Δ of table 1 indicates the difference between the mean value of the OD measured at 0 seconds of presence and the mean OD value measured at 275 seconds of presence between the sample H1, the rh DHODH and its substrate.

The symbol Δ represents the decrease in the OD between 0 and 275 seconds of presence, that is, the capacity of transforming the DHO into ORO by the effective activity of the DHODH.

In table 1, the last two rows give, for each sample concentration of H1, the percentage of activity of conversion of the colored DHO into ORO by the activity of the rh DHODH, as well as the percentage of inhibition of the activity of the rh DHODH by the sample H1.

The % of activity is calculated as follows, for a given sample concentration H1 (subsequently H2):

% of activity=(ΔH1×100)/Δ of the negative control.

For example, for sample H1 to 0.01 μg/mL: the % of activity=(0.092×100)/0.208=44.231.

The % of inhibition is calculated by the following formula: 100—the value of the % of activity.

To validate the effective activity of the rh DHODH of transforming its DHO substrate during the experiment, a negative control was carried out in duplicate. This negative control is essential to validate the activity of the DHODH on its substrate and to determine the % of activity and the % inhibition of the samples.

The negative control column shows the mean OD values measured, at 600 nm, at the various measurement intervals in seconds.

The negative control contains: rh DHODH denoted “E” in table 1 at a concentration of 0.06 μg/mL of the total solution in the well, with 50 μL of the mixture of colored substrate denoted “S” and, as a replacement for sample H1, only a solution of DMSO buffer denoted “T”.

The results show that between 0 and 275 seconds, the mean value of the OD measured at 600 nm decreases.

Consequently, the colored substrate DHO is indeed converted into ORO, by the reduction activity by the enzyme rh DHODH. The enzyme rh DHODH is indeed functional with respect to the substrate mixture. In addition, neither the DMSO buffer solution nor the test buffer solution in which the DHO substrate has been diluted has an impact on the dehydrogenation activity by the enzyme rh DHODH.

As can be seen in table 1, the sample H1 of the invention inhibits the activity of DHODH for its substrate. For all the H1 concentrations tested, the percentage of inhibition is between 51% and 56%. It is also found that for the concentrations tested, an increase in the concentration of the sample H1 is not synonymous with an increase in the value of the percentage inhibition against the activity of the DHODH.

The same protocol and the same OD measurements made it possible to quantify the percentage inhibition of the sample H2.

Table 2 below shows the results obtained for the various tested concentrations of sample H2

TABLE 2
Inhibition of the activity of the DHODH by sample H2

| | Concentration of the H1 samples in μg of | | | | | | |
|---|---|---|---|---|---|---|---|
| | H1/mL of total solution in the well | | | | | | Negative |
| Time | 0.01 | 0.1 | 1 | 10 | 100 | 1000 | control |
| (s) | μg/mL | μg/mL | μg/mL | μg/mL | μg/mL | μg/mL | E + S + T |
| 0 | 0.312 | 0.330 | 0.337 | 0.347 | 0.358 | 0.366 | 0.349 |
| 55 | 0.299 | 0.311 | 0.320 | 0.333 | 0.340 | 0.350 | 0.321 |
| 110 | 0.283 | 0.295 | 0.297 | 0.311 | 0.320 | 0.325 | 0.241 |
| 165 | 0.254 | 0.269 | 0.279 | 0.282 | 0.287 | 0.303 | 0.211 |
| 220 | 0.235 | 0.244 | 0.258 | 0.260 | 0.270 | 0.304 | 0.192 |
| 275 | 0.213 | 0.224 | 0.230 | 0.240 | 0.251 | 0.296 | 0.141 |
| Δ | 0.099 | 0.106 | 0.107 | 0.107 | 0.107 | 0.07 | 0.208 |
| % activity | 47.596 | 50.961 | 51.440 | 51.440 | 51.440 | 33.654 | 100.000 |
| % inhibition | 52.404 | 49.039 | 48.56 | 48.56 | 48.56 | 66.346 | 0.000 |

As can be seen in table 2, the sample H2 of the invention inhibits the activity of DHODH.

For all the H2 concentrations tested, the percentage of inhibition is between 48% and 67%.

Unlike sample H1, there appears to be a dose-response effect. Indeed, for a concentration of 1000 μg/mL, the percentage of inhibition appears to be significantly higher than for a concentration of 0.01 μg/mL.

In other words, when the concentration of the sample H2 is increased in the presence of the enzyme, the percentage of inhibition increases. Specimen H2 differs from sample H1 by the Neurolaena lobate leaf extract preparation protocol. It would therefore seem that the elements responsible for the inhibitory activity of rh DHODH be concentrated in the leaves and that, according to the mode of preparation of the mother tincture of leaves, the inhibitory activity is different.

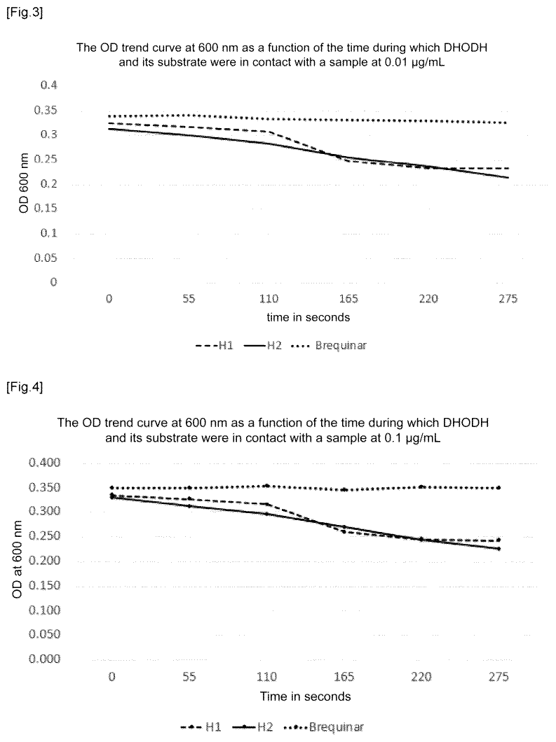

FIGS. 3 to 5 show the trend of the optical density measured at 600 nm over time, for samples of H1, H2 or brequinar at a given concentration.

The curve of FIG. 3 shows the measurement of the OD values at 600 nm, over time, for samples at a total concentration of 0.01 μg/mL in the well.

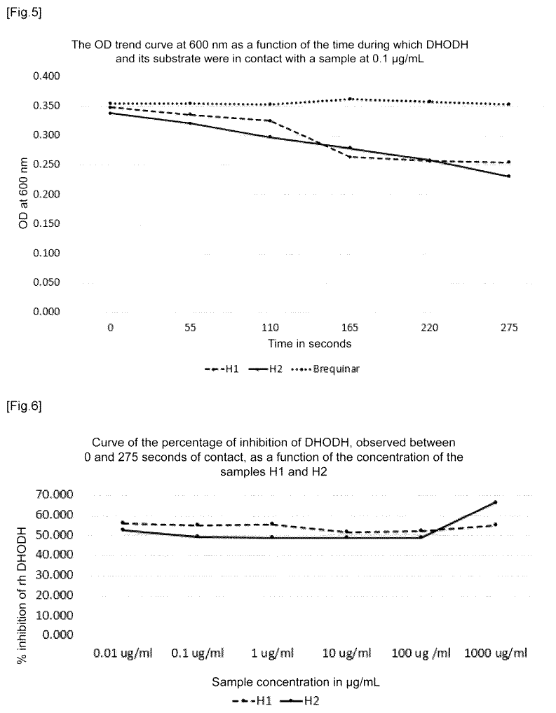

In the same way, the curve of FIG. 4 is for samples at a concentration of 0.1 μg/mL and the curve of FIG. 5 is for samples at a concentration of 1 μg/mL.

It should be noted that, in any case, regardless of the concentration of the sample, for the brequinar, a known inhibitor of DHODH, the OD at 600 nm remains virtually stable over time. The brequinar is therefore an inhibitor of the DHODH.

For samples H1 and H2, a reduction in the OD measured at 600 nm is observed. This decrease is synonymous with the activity of rh DHODH. However, it is found that for each of the samples, the reduction in the OD is clear from 110 seconds of being in contact.

Between 0 and 110 seconds, the OD measured at 600 nm is rather stable. This observation seems to mean that the DHODH enzyme is not active immediately. In the first 110 seconds, the DHODH does not perform the dehydrogenation reaction or does so very little, when it is in the presence of the samples H1 or H2. The samples H1 and H2 therefore seem to slow the start of the DHODH activity with respect to its substrate.

In order to effectively validate the enzyme rh DHODH's activity of reducing its substrate, a positive control was also carried out in duplicate. The positive control wells comprise, as replacement for the diluted sample H1 or H2, brequinar, which is a known inhibitor of the activity of the DHODH.

In parallel with the OD measurement of the samples, the OD of a solution of brequinar diluted in a DMSO buffer was measured in the presence of the rh DHODH, and of its substrate.

In the same way as for the samples, the action of different concentrations of the brequinar solution was measured on the inhibition of the rh DHODH.

In order to validate the activity of the rh DHODH on its substrate, a positive control is carried out in triplicate, in parallel with the protocol for measuring the inhibition of the DHODH by the brequinar. The positive control wells comprise the rh DHODH called “E”, and its mixture of substrate called “T” as well as, replacing the brequinar, the DMSO buffer called “T”.

Table 3 below shows the results obtained for the various tested concentrations of the brequinar sample.

TABLE 3
Inhibition of the activity of the DHODH by brequinar

| | Concentration of the brequinar samples in μg of | | | | |
|---|---|---|---|---|---|
| | brequinar/mL of total solution in the well | | | | Positive |
| Time | 0.001 | 0.01 | 0.1 | 1 | control |
| (s) | μg/mL | μg/mL | μg/mL | μg/mL | E + S + T |
| 0 | 0.339 | 0.344 | 0.349 | 0.354 | 0.349 |
| 55 | 0.340 | 0.345 | 0.348 | 0.354 | 0.321 |
| 110 | 0.333 | 0.339 | 0.353 | 0.352 | 0.241 |
| 165 | 0.331 | 0.336 | 0.345 | 0.361 | 0.211 |
| 220 | 0.328 | 0.348 | 0.350 | 0.357 | 0.192 |
| 275 | 0.326 | 0.329 | 0.349 | 0.353 | 0.141 |
| Δ | 0.013 | 0.015 | 0.000 | 0.001 | 0.208 |
| % activity | 6.250 | 7.211 | 0.000 | 0.000 | 100.000 |
| % inhibition | 93.750 | 92.789 | 100.000 | 99.999 | 0.000 |

In table 3, the activity of the rh DHODH of converting its substrate is validated by the positive control. Indeed, over time, a reduction in the OD measured at 600 nm is effectively observed. This decrease results in the conversion of DHO into ORO, by the dehydrogenation activity of rh DHODH. Neither the DMSO buffer nor the test buffer impact the activity of the rh DHODH enzyme. The rh DHODH enzyme is therefore functional in this protocol for measuring the inhibition of the DHODH by the brequinar.

Table 3 shows that the various concentrations of brequinar tested exhibit an inhibition activity of rh DHODH of between 92% and 100%.

To highlight the inhibition activity of the samples H1 and H2, FIG. 6 shows the curve of the percentage inhibition of the samples H1 and H2 relative to each other, as a function of their concentration, after 275 seconds of bringing the DHODH and its substrate into contact.

In FIG. 6, it is noted that, for concentrations ranging from 0.01 to 1000 μg/mL, H1 has a relatively stable percentage of inhibition of the rh DHODH.

On the contrary, H2 has a significant increase in its percentage inhibition for a concentration exceeding 100 μg/mL. In particular, for a concentration of 1000 μg of H2/mL of total solution in the well, the percentage inhibition of rh DHODH advantageously reaches 66.3%.

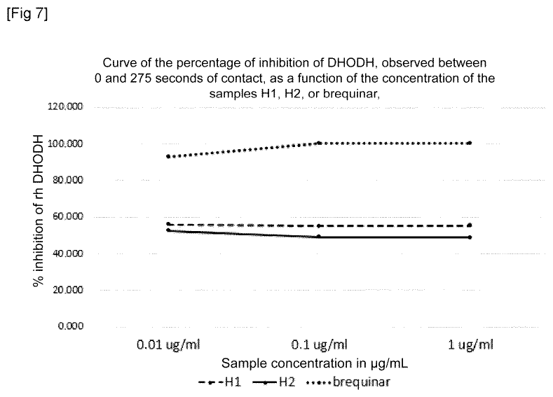

In the same way, FIG. 7 shows the evolution of the percentage inhibition of the H1, H2 and brequinar samples as a function of their concentration after 275 seconds of being in contact with the DHODH and its substrate. It is noted that the samples H1 and H2, just like the brequinar, are inhibitors of the activity of the DHODH on a substrate. Indeed, although the inhibition of DHODH by samples H1 and H2 is not 100% and as effective as the brequinar, it does exist.

Therefore, with regard to the results obtained, the samples of mother tinctures of Neurolaena lobata H1 and H2 of natural origin actually have, and significantly so, an inhibitory activity on the DHODH enzyme.

Thus, the therapeutic composition of the invention comprising a mother tincture of a leaf extract of a plant belonging to the genus “Neurolaena” and in this case “lobata” has the effect of inhibiting the activity of the DHODH.

As a result, a therapeutic composition comprising either of these samples, that is a leaf-based mother tincture of Neurolaena lobata is a promising, non-toxic, natural product for treating diseases whose therapeutic target is the inactivation of DHODH.

In particular, the therapeutic composition of the invention is a conceivable track for treating diseases resulting from infection by a viral pathogen, in particular RNA-genome viruses.

In vitro tests were also conducted to demonstrate the antiviral and virucidal efficacy, as well as to determine the inhibitory effect on the release of certain cytokines, of the mother tincture-based therapeutic composition of Neurolaena lobata.

Preparation of the Samples to be Tested:

Several samples were prepared from leaves of the plant Neurolaena lobata for conducting these tests.

The samples are denoted “TOTUM”.

The “TOTUM 3” corresponds to a mother tincture obtained from leaves of Neurolaena lobata; it was prepared in the following way:
i) A mass of 3.6 kg of dried leaves of Neurolaena lobata was mixed with a volume of 225 L sugar cane alcohol at 50°, which corresponds to a mass concentration of 16 g of dried leaves of Neurolaena lobata per liter of sugar cane alcohol; more generally, a mixture is prepared having a mass concentration of between 15 and 20 g/L, preferably between 16 and 17 g/L and, more preferably, equal to 16 g of dried leaves of Neurolaena lobata per liter of sugar cane alcohol at 50°;

Preferentially, as for the preparation of the sample H1 mentioned above, the leaves can be dried under a stream of hot air, at a temperature preferably below 40° C., for approximately 120 hours, until they have a residual moisture content on the order of 6 to 7%. The moisture content can be determined in the same way as for H1 as well.
ii) The mixture is left to macerate under stirring for approximately 21 days, at a temperature of the order of 30° C., with slow stirring every day for 12 h;
iii) After maceration, the mixture is filtered on “cartridges” or filtration pockets with a porosity of 50-75 μm and a filtrate and a retentate;
iv) a volume of 3 L of filtrate is collected
v) the alcohol contained therein is evaporated by a rotary evaporator, in three periods, until an aqueous solution has a volume approximately equal to 1 L.
vi) The aqueous solution thus obtained is frozen, then freeze-dried.

Finally, a lyophilisate having a mass equal to 12.3 g is obtained.

The “TOTUM 4” is a sample which is diluted from the mother tincture.

More particularly, in order to obtain this sample, in step iv) of the protocol for obtaining the TOTUM 3 below, instead of collecting 3 L of filtrate, 0.75 L of filtrate is collected which is diluted in a volume equal to 2.25 L of water. A hydro-alcoholic solution having a total volume equal to 3 L is then obtained. In general, the liquid filtrate, or mother tincture, obtained in step iii) above is diluted to ¼.

Then, the following steps for obtaining the TOTUM 4 are similar to those used to obtain the TOTUM 3 and which have been described above, namely:
v) the alcohol contained in the hydro-alcoholic solution, preferably having a volume equal to 3 L, is evaporated by a rotary evaporator, for example in three periods, until an aqueous solution is obtained, which may have a volume approximately equal to 1 L;
vi) The aqueous solution thus obtained is frozen, then freeze-dried.

Finally, a lyophilisate having a mass equal to 5.6 g is obtained, corresponding to a dry extract of a mother tincture diluted (to one-quarter) of dried leaves of Neurolaena lobate.

As a negative control, a sample called “TOTUM 2” was prepared from dried banana pulp Musa sapientum.

More particularly, for obtaining this sample, 150 g of dried banana pulp were diluted in a volume of 5 L sugar cane alcohol at 50°.

The mixture is macerated for a period of about 5 days, with stirring for 2 to 3 hours per day, before being filtered on paper with a porosity of between 10 and 20 μm, in order to obtain 4 L of hydro-alcoholic filtrate.

The filtrate is then evaporated by a rotary evaporator, until a dry extract is obtained, with a mass equal to 6 g.

Evaluation of the inhibition of the expansion of the SARS-CoV2 virus responsible for Covid-19 during the treatment of human lung epithelial cells (Calu-3) and of renal cells (VeroE6-TMPRSS2) by TOTUMs 2, 3 and 4

The SARS-CoV2 virus strain which was used during the driving of these tests is the European strain (a mutation of the original Wuhan strain in D614G), which corresponds to the SARS-CoV-2 strain denoted Slovakia/SK-BMCS/2020.

The viral strain was provided by the European Virus Archive goes Global (Evag) platform (https://www.european-virus-archive.com/).

The viral strain of SARS-Cov2 was amplified and titrated on the Vero E6 TMPRSS2 cell line by Oncodesign.

Two cell lines were used in these evaluation tests: these are the following line:
Calu-3, human lung adenocarcinoma (from ATCC—American Type Culture Collection);
Vero E6-TMPRSS2, non-human primate kidney epithelial cells (from NIBSC—National Institute for Biological Standards and Controls, UK).

The Calu-3 cell model is already well described in the literature for SARS-CoV (see C.-T. K. Tseng, J. Tseng, L. Perrone, M. Worthy, V. Popov, and C. J. Peters, “Apical entry and release of severe acute respiratory syndrome-associated coronavirus in polarized Calu-3 lung epithelial cells,” J Virol, vol. 79, no. 15, pp. 9470-9479, Aug. 2005, doi: 10.1128/JVI.79.15.9470-9479.2005).

The Calu-3 cells were cultured in a monolayer at 37° C. in a humidified atmosphere (5% CO2, 95% air) in the corresponding cell culture medium (MEM+1% pyruvate+1% glutamine+10% Fetal Bovine Serum).

The Vero E6-TMPRSS2 cells were cultured in a monolayer at 37° C. in a humidified atmosphere (5% CO2, 95% air) in the corresponding cell culture medium (DMEM+1% pyruvate+1% cocktail of antibiotics (penicillin, streptomycin and geneticin)+2% fetal bovine serum).

The cells of these two cell lines are adhered onto the plastic flasks. For cell passage procedures, the cells were detached from the culture bottle by a 20-minute treatment (for the cells of the Calu line) and 5 minutes (for the cells of the Vero line) with trypsin-versene and neutralized by adding a complete culture medium. For the study, the cells were deposited on 96-well plates.

The cells were counted and their viability was evaluated using the Vi-cell counter.

In a first series of tests, denoted “CAS1”, the cells of the two aforementioned cell lines are brought into contact with the compounds to be tested (TOTUM 2, 3 and 4 in particular), for a period of 24 h, before exposure to the viral strain of SARS-CoV-2. This first series “CAS 1” makes it possible to study the antiviral effect, in other words the cells are treated by the compound before being infected.

In a second series of tests, denoted “CAS 2”, the viral strain of SARS-CoV-2 is brought into contact with the various compounds to be tested, including TOTUMs 2, 3 and 4, for a period of 30 min at room temperature, before bringing the cells into contact with the virus. This second series “CAS 2” makes it possible to study the virucidal effect, in other words the virus is brought into contact with the compound before being brought into contact with the cells.

Test protocol for CAS 1 tests with the cell lines Calu-3 and Vero E6 TMPRSS2

The cells were counted and their viability evaluated using the cell analyzer Vi-CELL.

The cells were seeded to reach confluence:
Vero E6 TMPRSS2-30,000 cells/well;
Calu-3-90,000 cells/well.

From the lyophilisates and dry extracts of TOTUMs 2, 3 and 4, stock solutions are prepared in DMSO at 10 mg/mL. From these stock solutions, seven concentrations of test compounds were prepared in a complete growth medium and added to the cells: 10000, 3333, 1111, 370, 123, 41, 14 ng/mL.

The first biological replicate (N=1) was carried out with these concentrations.

The second biological replicate (N=2) was carried out with different concentrations. Indeed, the concentrations tested were adjusted after analyzing the results of the first biological replicate.

Thus, for the replicate N=2, the following concentrations were used: 100000, 33333, 11111, 3704, 1235, 412 and 137 ng/mL for the TOTUM 3 and 20000, 6667, 2222, 741, 247, 82 and 27 ng/mL for TOTUMs 2 and 4.

As a reference control compound, or positive control, the active metabolite of remdesivir was used. Seven remdesivir concentrations (20000, 6667, 2222, 741, 247, 82, 27 nM) were prepared and added to the cells.

The active metabolite of remdesivir was provided by Oncodesign, in the form of a 20 mM mother solution in DMSO.

The plates were incubated for 24 h at 37° C.

Next, a volume of 10 μL of viral preparation equivalent to a MOI (Multiplicity of Infection)=0.01 was added and incubated at 37° C. for 48 h for the VeroE6 TMPRSS2 cells and 72 h for the Calu-3 cells.

A fraction (50 μL) of the supernatants was collected and stored at a temperature equal to ?20° C. to determine the viral load.

A fraction (˜200 μL in three aliquots: 2×50 μL+the remaining volume) of the supernatants was collected and stored at a temperature equal to ?20° C. for the cytokine assay.

Test protocol for CAS 2 tests with the cell lines Calu-3 and Vero E6 TMPRSS2

The cells were counted and their viability was evaluated using the cell analyzer Vi-CELL.

The cells were seeded to reach confluence:
Vero E6 TMPRSS2-35,000 cells/well;
Calu-3-80,000 cells/well.

From the dry lyophilisates and extracts of TOTUMs 2, 3 and 4, seven concentrations of test compounds were prepared in a fresh growth medium: 10000, 3333, 1111, 370, 123, 41, 14 ng/mL.

The first biological replicate (N=1) was carried out with these concentrations.

Just as for CAS 1, the second biological replicate (N=2) was carried out with different concentrations.

Thus, for the replicate N=2, the following concentrations were used: 100000, 33333, 11111, 3704, 1235, 412 and 137 ng/mL for the TOTUM 3 and 20000, 6667, 2222, 741, 247, 82 and 27 ng/mL for TOTUMs 2 and 4.

Likewise, seven concentrations of the reference control, or positive control, the active metabolite of remdesivir (20000, 6667, 2222, 741, 247, 82, 27 nM) were prepared.

A volume of 10 μL of viral preparation equivalent to a MOI=0.01 was mixed with the test compounds and incubated at room temperature for 30 min.

The compound/virus mixture was then added to the cells.

The cells were incubated at 37° C. for 48 h for the Vero6-TMPRSS2 cells and 72 h for the Calu-3 cells.

A fraction (50 μL) of the supernatants was collected and stored at ?20° C. to determine the viral load.

A fraction (˜200 μL in three aliquots: 2×50 μL+the remaining volume) of the supernatants was collected and stored at ?20° C. for the cytokine assay.

Note: a plate without any virus was prepared in order to evaluate the cytotoxicity of the compounds tested on both cell types. The cell viability was evaluated by the CellTiter Glo test for all the conditions according to the manufacturer's recommendations (Promega, G7570).

For CAS 1 and CAS 2, the quantification of the viral load by RTqPCR, targeting the ORF1ab viral gene, was carried out at the end of the experiment.

The extraction of the viral RNA was carried out by the Macherey Nagel Viral RNA kit and the RNA was frozen at −80° C. until the RT-qPCR was carried out.

The complete RT-qPCR was carried out using the SuperScript™ On-Step qRT-PCR System kit, with primers and qRT-PCR conditions targeting the ORF1ab gene. The amplifications were carried out with a Bio-Rad CFX384™ apparatus and corresponding software.

Also for CAS 1 and CAS 2, the CellTiter-Glo® luminescent cell viability test or cytotoxicity test was carried out both on a control plate (without virus) and on the treated and infected plates to evaluate the cytotoxicity of the samples tested.

The CellTiter-Glo® luminescent cell viability test is a homogeneous method for determining the number of viable cells in culture based on the quantification of the ATP present, an indicator of metabolically active cells.

The method was used for the VeroE6-TMPRSS2 and Calu-3 cells in the absence of viruses to establish the cytotoxicity of each of the compounds that were tested.

The method was also used for the VeroE6-TMPRSS2 cells in the presence of viruses 48 hours after infection in the case of cytopathogenic effects (presence of active viruses); the presence of viruses in the Vero cell model results in cytopathogenic effects after use of the cellular machinery while the virus is continuously produced in the Calu model.

The test was carried out according to the supplier's protocol.

After removing the entire supernatant for the PCR reactions and the cytokine assays, add 100 μL of fresh cell medium to 100 μL of reagent and incubate until the luminescence is recorded, at least 15 min after mixing.

For CAS 1 and 2, the dosage of cytokines, more particularly IL 6, of MCP1 and IP10 were carried out by ELISA using commercial kits on cell culture supernatants collected 48 hours and 72 hours post-infection, respectively for the Vero cell lines and Calu-3.

Results: evaluation of the cytotoxicity of the TOTUMs (without virus)

The toxicity of the “TOTUMs” test samples and the active metabolite of remdesivir on Calu-3 cells not exposed to the virus was evaluated by measuring cell viability after 96 hours of exposure to the compounds.

The results are presented in the tables below, the cell viability being expressed as a % relative to the untreated cells, after exposure to compounds of the cell line Calu-3, with SD corresponding to the standard deviation:

Active metabolite of remdesivir

| (nM) | Avg | SD |
|---|---|---|
| 27 | 91 | 17 |
| 82 | 94 | 14 |
| 247 | 116 | 22 |
| 741 | 103 | 34 |
| 2222 | 111 | 20 |
| 6667 | 96 | 11 |
| 20000 | 116 | 18 |

| | | | |
|---|---|---|---|
| cc | TOTUM 2 | TOTUM 3 | TOTUM 4 |

| (ng/mL) | Avg | SD | Avg | SD | Avg | SD |
|---|---|---|---|---|---|---|
| 14 | 131 | 11 | 120 | 8 | 102 | 31 |
| 41 | 93 | 20 | 64 | 18 | 109 | 23 |
| 123 | 96 | 4 | 111 | 17 | 106 | 25 |
| 370 | 99 | 51 | 110 | 4 | 138 | 2 |
| 1111 | 103 | 39 | 90 | 4 | 103 | 31 |
| 3333 | 124 | 25 | 99 | 11 | 120 | 29 |
| 10000 | 113 | 20 | 95 | 7 | 110 | 22 |

After treatment with the active metabolite of remdesivir, the results are similar between the biological replicates with an average cell viability ranging from 91% (27 nM, N=1) to 116% (247 nM and 20000 nM, N=1).

During the biological replicate N=1, the cell viability after treatment with the TOTUMS 2, 3 and 4, for seven concentrations ranging from 14 ng/mL to 10,000 ng/mL, was similar to that obtained after treatment with the active metabolite of remdesivir.

Similar results were also obtained for TOTUMs 2 and 4 during the biological replicate N=2 (see tables below), for the concentrations tested ranging from 137 ng/mL to 100,000 ng/mL.

Active metabolite of remdesivir

| (nM) | Avg | SD |
|---|---|---|
| 27 | 112 | 3 |
| 82 | 108 | 2 |
| 247 | 109 | 5 |
| 741 | 106 | 2 |
| 2222 | 113 | 2 |
| 6667 | 113 | 1 |
| 20000 | 104 | 2 |

| | |
|---|---|
| cc | TOTUM 3 |

| (ng/mL) | Avg | SD |
|---|---|---|
| 137 | 102 | 3 |
| 412 | 93 | 3 |
| 1,235 | 98 | 3 |
| 3,704 | 99 | 5 |
| 11,111 | 96 | 4 |
| 33,333 | 84 | 3 |
| 100,000 | 1 | 0 |

| | | |
|---|---|---|
| cc | TOTUM 2 | TOTUM 4 |

| (ng/mL) | Avg | SD | Avg | SD |
|---|---|---|---|---|
| 27 | 106 | 4 | 105 | 1 |
| 82 | 101 | 4 | 96 | 2 |
| 247 | 104 | 3 | 101 | 4 |
| 741 | 104 | 1 | 103 | 4 |
| 2,222 | 103 | 4 | 100 | 3 |
| 6,667 | 103 | 4 | 102 | 2 |
| 20,000 | 97 | 2 | 99 | 0 |

However, it should be noted that a reduction in cell viability was observed for the TOTUM 3, for a concentration of 100,000 ng/mL.

The toxicity of TOTUMs 2, 3 and 4 and the active metabolite of remdesivir on VeroE6-TMPRSS2 cells not exposed to the virus was also evaluated by measuring cell viability after 72 hours of exposure to the compounds.

The results are presented in the tables below, the cell viability being expressed as a % relative to the untreated cells, after exposure to compounds of the cell line Vero E6, with SD corresponding to the standard deviation:

| | |
|---|---|
| Active metabolite | Active metabolite |
| of remdesivir | of remdesivir |

| (nM) | Avg | SD | (nM) | Avg | SD |
|---|---|---|---|---|---|
| 27 | 92 | 2 | 27 | 104 | 4 |
| 82 | 95 | 2 | 82 | 106 | 4 |
| 247 | 93 | 2 | 247 | 105 | 2 |
| 741 | 92 | 5 | 741 | 105 | 1 |
| 2222 | 94 | 5 | 2222 | 105 | 1 |
| 6667 | 93 | 5 | 6667 | 106 | 1 |
| 20000 | 97 | 3 | 20000 | 109 | 5 |

After treatment with the active metabolite of remdesivir, the results are similar between the biological replicates with an average cell viability ranging from 92% (27 nM, N=1, table at left above) to 109% (20,000 nM, N=2, table at right).

During N=1, the cell viability after treatment with the TOTUMs 2, 3 and 4, for seven concentrations ranging from 14 ng/mL to 10,000 ng/mL, was similar to the ones obtained after treatment with the active metabolite of remdesivir.

| | | | |
|---|---|---|---|
| cc | TOTUM 2 | TOTUM 3 | TOTUM 4 |

| (ng/mL) | Avg | SD | Avg | SD | Avg | SD |
|---|---|---|---|---|---|---|
| 14 | 91 | 1 | 96 | 4 | 97 | 2 |
| 41 | 89 | 2 | 94 | 6 | 93 | 0 |
| 123 | 89 | 2 | 95 | 3 | 96 | 3 |
| 370 | 88 | 1 | 92 | 4 | 98 | 3 |
| 1111 | 89 | 1 | 93 | 2 | 96 | 2 |
| 3333 | 89 | 0 | 93 | 2 | 97 | 1 |
| 10000 | 90 | 1 | 91 | 1 | 111 | 1 |

Similar results were obtained for TOTUMs 2 and 4 during N=2 for the concentrations tested ranging from 137 ng/mL to 100,000 ng/mL.

| | |
|---|---|
| cc | TOTUM 3 |

| (ng/mL) | Avg | SD |
|---|---|---|
| 137 | 97 | 2 |
| 412 | 97 | 2 |
| 1,235 | 99 | 4 |
| 3,704 | 98 | 6 |
| 11,111 | 95 | 9 |
| 33,333 | 32 | 3 |
| 100,000 | 1 | 0 |

| | | |
|---|---|---|
| cc | TOTUM 2 | TOTUM 4 |

| (ng/mL) | Avg | SD | Avg | SD |
|---|---|---|---|---|
| 27 | 100 | 1 | 95 | 1 |
| 82 | 100 | 1 | 96 | 2 |
| 247 | 101 | 1 | 97 | 1 |
| 741 | 100 | 1 | 97 | 2 |
| 2,222 | 100 | 1 | 98 | 1 |
| 6,667 | 100 | 1 | 103 | 2 |
| 20,000 | 100 | 1 | 109 | 4 |

Moreover, for TOTUM 3, at concentrations of 33,333 ng/mL and 100,000 ng/mL, a cytotoxicity is present.

Results: CAS 1—Anti-viral effect of compounds

The objective of these tests is to evaluate the antiviral effect of compounds that must be analyzed, in other words, the cells are treated with the compound before being infected.

First, on the cell line Calu-3, the viral load was evaluated by quantification of the viral RNA by targeting the ORF1ab gene. The infected cell control is 100% the reference.

After treatment with the active metabolite of remdesivir, the results are similar between the biological replicates. The viral load decreases when the concentration of compound increases, with:
In N=1, a percentage relative to the infected and untreated cells ranging from 84% for 27 nM of remdesivir to 0% (undetectable viral load) for 2,222 nM, 6,667 nM and 20,000 nM,

Active metabolite of remdesivir

| (nM) | Avg | SD |
|---|---|---|
| 27 | 84 | 80 |
| 82 | 107 | 4 |
| 247 | 93 | 43 |
| 741 | 22 | 6 |
| 2222 | 0 | 0 |
| 6667 | 0 | 0 |
| 20000 | 0 | 0 |

In N=2, a percentage relative to the infected and untreated cells ranging from 92% for 55 nM of remdesivir to 0% (undetectable viral load) for 4,444 nM, 13,333 nM and 40,000 nM.

Active metabolite of remdesivir

| (nM) | Avg | SD |
|---|---|---|
| 55 | 92 | 6 |
| 165 | 85 | 12 |
| 494 | 62 | 6 |
| 1481 | 6 | 4 |
| 4444 | 0 | 0 |
| 13333 | 0 | 0 |
| 40000 | 0 | 0 |

For the compounds tested, during N=1, the viral load ranged from 67% (TOTUM 3 at 370 ng/mL) to 280% (TOTUM 2 at 10,000 ng/mL).

| | | | |
|---|---|---|---|
| cc | TOTUM 2 | TOTUM 3 | TOTUM 4 |

| (ng/mL) | Avg | SD | Avg | SD | Avg | SD |
|---|---|---|---|---|---|---|
| 14 | 83 | 6 | 146 | 89 | 83 | 38 |
| 41 | 85 | 18 | 88 | 16 | 80 | 15 |
| 123 | 95 | 6 | 97 | 37 | 79 | 23 |
| 370 | 75 | 9 | 67 | 20 | 80 | 8 |
| 1111 | 81 | 20 | 77 | 5 | 74 | 7 |
| 3333 | 151 | 51 | 96 | 221 | 72 | 10 |
| 10000 | 280 | 62 | 151 | 23 | 122 | 19 |

During N=2, results similar to N=1 were observed for the TOTUM 2, for seven concentrations. For the TOTUM 4 at 6,667 ng/mL and 20,000 ng/mL a viral load of 59% and 49% was observed.

| | |
|---|---|
| TOTUM 2 | TOTUM 4 |

| (ng/mL) | Avg | SD | Avg | SD |
|---|---|---|---|---|
| 27 | 90 | 10 | 83 | 3 |
| 82 | 102 | 31 | 91 | 8 |
| 247 | 89 | 4 | 80 | 22 |
| 741 | 97 | 14 | 85 | 12 |
| 2222 | 111 | 24 | 67 | 10 |
| 6667 | 105 | 5 | 59 | 7 |
| 20000 | 96 | 16 | 49 | 12 |

Moreover, for TOTUM 3 at 100,000 ng/mL an undetectable viral load (0%) was observed.

TOTUM 3

| (ng/mL) | Avg | SD |
|---|---|---|
| 137 | 114 | 28 |
| 412 | 72 | 26 |
| 1235 | 66 | 17 |
| 3704 | 67 | 14 |
| 11111 | 54 | 21 |
| 33333 | 113 | 43 |
| 100000 | 0 | 0 |

A dosage of three cytokines (IL6, IP10 and MCP1) was carried out on the cell culture supernatants (in ng/mL) on the Calu 3 lung cell line inoculated with SARS-CoV-2. The cells were treated with the products tested for 24 hours and then inoculated with the viral strain for 72 hours. The concentrations of compounds are indicated in nM for the active metabolite of remdesivir and in ng/mL for the tested TOTUMs.

IL6 is a pro-inflammatory cytokine, expressed at a basal rate of 300 μg/mL. In the event of infection, its rate is greatly increased on the order of 2,000 μg/mL. The chemokine IP10 is involved in inflammatory processes, undetectable in basal rate. In the event of infection, its rate is greatly increased on the order of 400 μg/mL.

The cytokine MCP1 could not be detected in the studies carried out. The cytokines have a transient expression; therefore, at the moment when the assay is carried out, the cytokine has already been expressed or will be subsequently expressed, given that a single read point is carried out, respectively at 48 h and 72 h post-infection for the Vero and Calu models, and not a kinetic read.

For IL6 (table below to the left for the first replicate N=1 and to the right for the second replicate N=2), a dose-response effect was observed for the active metabolite of remdesivir as expected, with decreasing concentrations of cytokines when the concentrations of compound increased.

| | |
|---|---|
| Active metabolite | Active metabolite |
| of remdesivir | of remdesivir |

| (nM) | Avg | SD | (nM) | Avg | SD |
|---|---|---|---|---|---|
| 27 | 928 | 212.7 | 55 | 680 | 106.6 |
| 82 | 771 | 60.6 | 165 | 631 | 62.0 |
| 247 | 651 | 35.2 | 494 | 508 | 21.0 |
| 741 | 586 | 280.9 | 1481 | 301 | 23.0 |
| 2222 | 255 | 26.1 | 4444 | 221 | 4.8 |
| 6667 | 273 | NA | 13333 | 172 | 15.1 |
| 20000 | 308 | 49.1 | 40000 | 245 | 57.1 |

For the chemokine IP10 (table below to the left for replicate N=1 and to the right for replicate N=2) a dose-response effect is also observed:

| | |
|---|---|
| Active metabolite | Active metabolite |
| of remdesivir | of remdesivir |

| (nM) | Avg | SD | (nM) | Avg | SD |
|---|---|---|---|---|---|
| 27 | 291 | 94 | 55 | 462 | 26 |
| 82 | 233 | 10 | 165 | 422 | 96 |
| 247 | 205 | 2 | 494 | 382 | 17 |
| 741 | 162 | 106 | 1481 | 62 | 61 |
| 2222 | NA | NA | 4444 | NA | NA |
| 6667 | NA | NA | 13333 | NA | NA |
| 20000 | NA | NA | 40000 | NA | NA |

It is particularly interesting to note that similar results, namely a dose-response effect, were observed for the TOTUMs 3 and 4 for N=1 during the assay of the IL6.

| | |
|---|---|
| TOTUM3 | TOTUM4 |

| (ng/mL) | Avg | SD | (ng/mL) | Avg | SD |
|---|---|---|---|---|---|
| 14 | 1696 | 762.1 | 14 | 886 | 92 |
| 41 | 1097 | 543.4 | 41 | 710 | 44 |
| 123 | 882 | 324.5 | 123 | 788 | 13 |
| 370 | 828 | 363.6 | 370 | 674 | 150 |
| 1111 | 655 | 231.7 | 1111 | 654 | 95 |
| 3333 | 650 | 220.0 | 3333 | 468 | 26 |
| 10000 | 478 | 51.1 | 10000 | 767 | 162 |

The same applies for N=2.

| | |
|---|---|
| TOTUM 3 | TOTUM 4 |

| (ng/mL) | Avg | SD | (ng/mL) | Avg | SD |
|---|---|---|---|---|---|
| 137 | 688 | 189.9 | 27 | 1721 | 1059 |
| 412 | 516 | 222.7 | 82 | 206 | 96 |
| 1235 | 463 | 73.1 | 247 | 247 | 95 |
| 3704 | 340 | 141.7 | 741 | 232 | 82 |
| 11111 | 174 | 77.4 | 2222 | 183 | 44 |
| 33333 | 42 | 32.9 | 6667 | NA | NA |
| 100000 | 117 | 20.1 | 20000 | 118 | 72 |

A dose-response effect is also remarkable during tests with the TOTUMs 3 and 4 for the N=1 for the dosage of the IP10, for the replicate N1:

| | |
|---|---|
| TOTUM 3 | TOTUM 4 |

| (ng/mL) | Avg | SD | (ng/mL) | Avg | SD |
|---|---|---|---|---|---|
| 14 | 511 | 241 | 14 | 231 | 36 |
| 41 | 325 | 190 | 41 | 166 | 12 |
| 123 | 225 | 101 | 123 | 180 | 6 |
| 370 | 211 | 129 | 370 | 173 | 73 |
| 1111 | 182 | 89 | 1111 | 167 | 38 |
| 3333 | 179 | 74 | 3333 | 103 | 1 |
| 10000 | 91 | 6 | 10000 | 178 | 37 |

The same applies for N=2:

| | |
|---|---|
| TOTUM 3 | TOTUM 4 |

| (ng/mL) | Avg | SD | (ng/mL) | Avg | SD |
|---|---|---|---|---|---|
| 137 | 363 | 99 | 27 | 470 | 208 |
| 412 | 273 | 110 | 82 | 340 | 43 |
| 1235 | 215 | 85 | 247 | 350 | 61 |
| 3704 | 149 | 79 | 741 | 406 | 59 |
| 11111 | NA | NA | 2222 | 360 | 50 |
| 33333 | NA | NA | 6667 | 165 | 82 |
| 100000 | NA | NA | 20000 | 117 | 10 |

No dose-response effect is observed for the TOTUM 2, whether in the context of the dosage of IL6 or in the context of the dosage of IP10 (results not shown).

On the cell line VeroE6-TMPRSS2, the viral load was evaluated by quantification of the viral RNA by targeting the ORF1ab gene.

After treatment with the active metabolite of remdesivir, the results are similar between the biological replicates. The viral load decreases when the concentration of compound increases, with:
In N=1, a percentage relative to the infected and untreated cells ranging from 101% for 27 nM of remdesivir to 0% (undetectable viral load) for 6,667 nM and 20,000 nM,

Active metabolite of remdesivir

| (nM) | Avg | SD |
|---|---|---|
| 27 | 101 | 15 |
| 82 | 122 | 24 |
| 247 | 120 | 14 |
| 741 | 148 | 14 |
| 2222 | 67 | 27 |
| 6667 | 0 | 0 |
| 20000 | 0 | 0 |

In N=2, a percentage relative to the infected and untreated cells ranging from 70% for 27 nM of remdesivir to 0% (undetectable viral load) for 6,667 nM and 20,000 nM:

Active metabolite of remdesivir

| (nM) | Avg | SD |
|---|---|---|
| 27 | 70 | 20 |
| 82 | 114 | 44 |
| 247 | 105 | 21 |
| 741 | 144 | 16 |
| 2222 | 7 | 3 |
| 6667 | 0 | 0 |
| 20000 | 0 | 0 |

During N=1, the viral load relative to the infected, untreated cells after treatment with the TOTUMs 2, 3 and 4, for seven concentrations ranging from 14 ng/mL to 10,000 ng/mL was similar or greater than that obtained after treatment at the lowest dose of active metabolite of remdesivir; the antiviral activity does not appear to be reduced.

These results are not described here.

During N=2, results similar to N=1 were observed for TOTUM 2 for seven concentrations ranging from 137 ng/mL to 100,000 ng/mL (no induced decrease in the viral load, therefore no antiviral activity of TOTUM 2).

Furthermore:
After treatment with TOTUM 3 at the concentrations of 33,333 ng/mL and 100,000 ng/mL, viral loads of 9% and 0%, respectively, were observed, in other words undetectable:

| | |
|---|---|
| cc | TOTUM 3 |

| (ng/mL) | Avg | SD |
|---|---|---|
| 137 | 129 | 13 |
| 412 | 105 | 14 |
| 1235 | 120 | 18 |
| 3704 | 102 | 17 |
| 11111 | 71 | 10 |
| 33333 | 9 | 7 |
| 100000 | 0 | 0 |

After treatment with TOTUM 4 at 6,667 ng/mL, a viral load of 78% relative to the untreated infected cells is observed and, for the treatment with the concentration of 20,000 ng/mL, a reduced viral load at 40% was observed:

TOTUM 4

| (ng/mL) | Avg | SD |
|---|---|---|
| 27 | 97 | 15 |
| 82 | 53 | 32 |
| 247 | 96 | 11 |
| 741 | 94 | 7 |
| 2222 | 95 | 12 |
| 6667 | 78 | 8 |
| 20000 | 40 | 5 |

The TOTUM 3 induces a significant reduction in the viral load, or even makes it undetectable, at concentrations of 33,333 ng/mL and 100,000 ng/mL.

The TOTUM 4 induces a substantial decrease in the viral load at the concentration of 20,000 ng/mL.

Results: CAS 2—Virucidal effect of the compounds

The goal of these tests is the evaluation of the virucidal effect of compounds that must be analyzed, in other words the virus was incubated with the compounds for 30 minutes before the cells are cultured with the pre-treated inocula.

First, on the cell line Calu-3, the viral load was evaluated by quantification of the viral RNA by targeting the ORF1ab gene.

After treatment with the active metabolite of remdesivir, the results are similar between the biological replicates. The viral load decreases when the concentration of compound increases, with:
In N=1, a percentage relative to the infected and untreated cells ranging from 113% for 27 nM of remdesivir to 0% for 6,667 nM and 20,000 nM:

Active metabolite of remdesivir

| (nM) | Avg | SD |
|---|---|---|
| 27 | 113 | 32 |
| 82 | 93 | 36 |
| 247 | 82 | 22 |
| 741 | 22 | 5 |
| 2222 | 1 | 1 |
| 6667 | 0 | 0 |
| 20000 | 0 | 0 |

In N=2, a percentage relative to the infected and untreated cells ranging from 109% for 27 nM of remdesivir to 0% for 2222 nM, 6667 nM and 20,000 nM:

Active metabolite of remdesivir

| (nM) | Avg | SD |
|---|---|---|
| 27 | 109 | 68 |
| 82 | 66 | 26 |
| 247 | 56 | 52 |
| 741 | 10 | 4 |
| 2222 | 0 | 0 |
| 6667 | 0 | 0 |
| 20000 | 0 | 0 |

During N=1, the viral load relative to the infected, untreated cells after treatment with the TOTUMs 2, 3 and 4, for seven concentrations ranging from 14 ng/mL to 10,000 ng/mL was similar or greater than that obtained after treatment at the lowest dose of active metabolite of remdesivir (results not shown).

During N=2, results similar to N=1 were observed for TOTUM 2 for seven concentrations ranging from 27 ng/mL to 20,000 ng/mL and from 14 ng/mL to 10,000 ng/mL.

Furthermore:
After treatment with TOTUM 3 at the concentration of 100,000 ng/mL a viral load of 0% (undetectable viral load) was observed;
After treatment with TOTUM 4 at the concentrations of 2,222 ng/mL and 6,667 ng/mL, viral loads of 46% and 43%, respectively, were observed:

TOTUM 4

| (ng/mL) | Avg | SD |
|---|---|---|
| 27 | 84 | 13 |
| 82 | 62 | 14 |
| 247 | 68 | 13 |
| 741 | 76 | 14 |
| 2222 | 46 | 7 |
| 6667 | 43 | 6 |
| 20000 | 90 | 8 |

A dosage of three cytokines was carried out for IL6 and IP10 on samples of cell culture supernatant.

For IL6, a dose-response effect was observed for the active metabolite of remdesivir with decreasing cytokine concentrations when the compound concentrations increased, for the replicate N=1 (table below to the left) and for the replicate N=2 (table below).

| | Active metabolite | | | Active metabolite | |
|---|---|---|---|---|---|
| | of remdesivir | | | of remdesivir | |
| (nM) | Avg | SD | (nM) | Avg | SD |
| 27 | 1617 | 232.5 | 55 | 738 | 25.5 |
| 82 | 1576 | 5.7 | 165 | 657 | 46.9 |
| 247 | 1362 | 180.3 | 494 | 680 | 72.5 |
| 741 | 966 | 33.8 | 1481 | 293 | 13.1 |
| 2222 | 169 | 6.2 | 4444 | 186 | 85.2 |
| 6667 | 154 | 88.2 | 13333 | 224 | 4.4 |
| 20000 | 203 | 55.3 | 40000 | 286 | 13.1 |

In the context of the assay of IL6, similar results were observed for TOTUMs 3 and 4 for N=1:

| | |
|---|---|
| TOTUM 3 | TOTUM 4 |

| (ng/mL) | Avg | SD | (ng/mL) | Avg | SD |
|---|---|---|---|---|---|
| 14 | 1917 | 319.6 | 14 | 1269 | 238 |
| 41 | 1535 | 296.9 | 41 | 1369 | 63 |
| 123 | 1643 | 619.3 | 123 | 1041 | 275 |
| 370 | 1670 | 645.1 | 370 | 1082 | 185 |
| 1111 | 1643 | 658.5 | 1111 | 1157 | 65 |
| 3333 | 1339 | 376.6 | 3333 | 1041 | 126 |
| 10000 | 625 | 12.3 | 10000 | 920 | 69 |

The same applies for replicate N=2:

| | |
|---|---|
| TOTUM 3 | TOTUM 4 |

| (ng/mL) | Avg | SD | (ng/mL) | Avg | SD |
|---|---|---|---|---|---|
| 137 | 867 | 163.0 | 27 | 581 | 125 |
| 412 | 803 | 305.2 | 82 | 544 | 37 |
| 1235 | 676 | 137.0 | 247 | 684 | 119 |
| 3704 | 523 | 118.0 | 741 | 578 | 68 |
| 11111 | 204 | 38.0 | 2222 | 475 | 11 |
| 33333 | 35 | 23.3 | 6667 | 297 | 31 |
| 100000 | 162 | 54.0 | 20000 | 227 | 54 |

For IP10, a dose-response effect is also to be noted when the virus is incubated with remdesivir for replicate N=1 (table below to the left) and for replicate N=2 (table below to the right).

| | Active metabolite | | | Active metabolite | |
|---|---|---|---|---|---|
| | of remdesivir | | | of remdesivir | |
| (nM) | Avg | SD | (nM) | Avg | SD |
| 27 | 410 | 42 | 55 | 123 | 28 |
| 82 | 405 | 11 | 165 | 129 | 15 |
| 247 | 309 | 2 | 494 | 133 | 38 |
| 741 | 276 | 11 | 1481 | 48 | 6 |
| 2222 | 21 | 14 | 4444 | 6 | 1 |
| 6667 | 27 | 34 | 13333 | 3 | 4 |
| 20000 | 9 | 5 | 40000 | 17 | 6 |

Similar results, namely a dose-response effect, were observed for TOTUMs 3 and 4 for the N=1 during the dosage of IP1O:

| | |
|---|---|
| TOTUM 3 | TOTUM 4 |

| (ng/mL) | Avg | SD | (ng/mL) | Avg | SD |
|---|---|---|---|---|---|
| 14 | 555 | 82 | 14 | 393 | 98 |
| 41 | 418 | 52 | 41 | 435 | 50 |
| 123 | 437 | 82 | 123 | 338 | 74 |
| 370 | 436 | 173 | 370 | 344 | 80 |
| 1111 | 385 | 134 | 1111 | 344 | 24 |
| 3333 | 327 | 91 | 3333 | 285 | 18 |
| 10000 | 95 | 28 | 10000 | 168 | 24 |

The same applies for replicate N=2:

| | |
|---|---|
| TOTUM 3 | TOTUM 4 |

| (ng/mL) | Avg | SD | (ng/mL) | Avg | SD |
|---|---|---|---|---|---|
| 137 | 116 | 36 | 27 | 110 | 31 |
| 412 | 139 | 39 | 82 | 126 | 4 |
| 1235 | 112 | 40 | 247 | 163 | 10 |
| 3704 | 79 | 5 | 741 | 153 | 7 |
| 11111 | 29 | 5 | 2222 | 108 | 2 |
| 33333 | 8 | 5 | 6667 | 46 | 10 |
| 100000 | 5 | 5 | 20000 | 20 | 2 |

Now on the cell line VeroE6-TMPRSS2, the viral load was evaluated by quantification of the viral RNA by targeting the ORF1ab gene.

After treatment with the active metabolite of remdesivir, the results are similar between the biological replicates. The viral load decreases when the concentration of compound increases, with:
In N=1, a percentage relative to the uninfected and untreated cells ranging from 97% for 27 nM of remdesivir to 0% and 1% (undetectable viral load) for 6,667 nM and 20,000 nM, respectively:

Active metabolite of remdesivir

| (nM) | Avg | SD |
|---|---|---|
| 27 | 97 | 12 |
| 82 | 122 | 7 |
| 247 | 109 | 9 |
| 741 | 114 | 9 |
| 2222 | 54 | 1 |
| 6667 | 0 | 0 |
| 20000 | 1 | 1 |

In N=2, a percentage relative to the uninfected and untreated cells ranging from 100% for 27 nM of remdesivir to 0% (undetectable viral load) for 2,222 nM, 6,667 nM and 20,000 nM,

Active metabolite of remdesivir

| (nM) | Avg | SD |
|---|---|---|
| 27 | 100 | 14 |
| 82 | 126 | 15 |
| 247 | 113 | 11 |
| 741 | 73 | 3 |
| 2222 | 0 | 0 |
| 6667 | 0 | 0 |
| 20000 | 0 | 0 |

During N=1, the viral load relative to the infected, untreated cells after treatment with the TOTUMs 2, 3 and 4, for seven concentrations ranging from 14 ng/mL to 10,000 ng/mL was similar or greater than that obtained after treatment at the lowest dose of active metabolite of remdesivir (results not shown, assumed to have no virucidal effect).

During N=2, results similar to N=1 were observed for the TOTUM 2 for seven concentrations ranging from 27 ng/mL to 20,000 ng/mL (results not shown, no virucidal effect of the TOTUM 2).

Furthermore:
After treatment with the TOTUM 3 at the concentration of 33333 ng/mL, a viral load of 16% is observed; at a concentration of 100,000 ng/mL, an undetectable viral load (0%) was observed:

TOTUM 3

| (ng/mL) | Avg | SD |
|---|---|---|
| 137 | 84 | 5 |
| 412 | 93 | 15 |
| 1235 | 106 | 6 |
| 3704 | 90 | 20 |
| 11111 | 77 | 8 |
| 33333 | 16 | 13 |
| 100000 | 0 | 0 |

After treatment with TOTUM 4 at the concentration of 20,000 ng/mL a viral load of 47% was observed:

| | | |
|---|---|---|
| cc | TOTUM 2 | TOTUM 4 |

| (ng/mL) | Avg | SD | Avg | SD |
|---|---|---|---|---|
| 27 | 103 | 17 | 106 | 10 |
| 82 | 115 | 15 | 117 | 30 |
| 247 | 106 | 11 | 111 | 11 |
| 741 | 128 | 11 | 108 | 17 |
| 2222 | 139 | 17 | 115 | 10 |
| 6667 | 148 | 27 | 90 | 0 |
| 20000 | 143 | 14 | 47 | 24 |

### CONCLUSIONS

The results that were obtained make it possible to demonstrate the following:

On the one hand, the compound remdesivir that was tested in vitro as a reference active metabolite against the SARS-CoV-2 virus clearly shows antiviral and virucidal activity against said virus, without any apparent cell toxicity. The viral load decreases when the remdesivir concentration increases for CAS 1 and CAS 2. Furthermore, for the cytokines IL6 and IP10, a dose-response effect is observed for the active metabolite of remdesivir, with decreasing concentrations in cytokines when the concentrations of said metabolite increase, as expected. These results make it possible to validate the test protocols used.

That said, remdesivir, although it has an interesting activity in vitro on the SARS-CoV-2 virus, also has notable nephrotoxic effects which may prove to be harmful for a patient suffering from Covid.

The sample referenced TOTUM 2 was obtained from dried banana pulp. The results obtained during the tests carried out demonstrate that such an extract does not exhibit any antiviral or virucidal effect. Furthermore, no dose-response effect on the release of cytokines could be observed for this sample.

Here again, these test results, expected to be negative, reinforce the protocol that was implemented.

With regard to the sample named TOTUM 3, it is obtained from a mother tincture of concentrated Neurolaena lobate, while the sample referenced under the name of TOTUM 4 corresponds to a dilution of the mother tincture which made it possible to also obtain said TOTUM 3, as emerges from the detailed description of the protocol for obtaining these TOTUMs 3 and 4 described above.

The results obtained with TOTUM 3 and detailed above demonstrate, on the one hand, that for compound concentrations ranging from 14 to 10,000 ng/mL, no toxicity is to be taken on the Calu-3 and Vero E6 cells.

However, there is notable cytotoxicity for a concentration of 100,000 ng/mL on the cells of the Calu-3 line. The same applies for the concentrations of 33,333 ng/mL and 100,000 ng/mL on the cells of the Vero E6 line, where cytotoxicity is present.

In parallel, for the TOTUM 3, at a concentration of 100,000 ng/mL, an undetectable viral load is observed for the cell line Calu-3. That said, this concentration was demonstrated as having cytotoxic effects. For the Vero E6 cell line, undetectable viral loads were observed for concentrations of 33,333 ng/mL and 100,000 ng/mL. Thus, again, a cytotoxicity is present.

It seems that the concentration of active compound in this sample is too great and leads to cytotoxicity on the cells.

The results obtained with the TOTUM 4, consisting of a dilution of the mother tincture to one-quarter, relative to the TOTUM 3 mentioned above, are, for their part, particularly advantageous.

For a memory, in the sample preparation method, according to the step of filtration of the mother tincture, in step iv) of the protocol for obtaining the TOTUM 3, 0.75 L of filtrate is withdrawn in a volume equal to 2.25 L of water. A hydro-alcoholic solution having a total volume equal to 3 L is then obtained, corresponding to a ¼ dilution of the mother tincture of dried leaves of Neurolaena lobate.

The results obtained with this TOTUM 4 demonstrate, on the one hand, that it does not exhibit any cytotoxicity on the model cell lines, regardless of the concentration tested, even for the highest ones.

In parallel with this lack of cytotoxicity, an antiviral activity of the TOTUM 4 is demonstrated for concentrations 6,667 ng/mL and 20,000 ng/mL, for which the viral loads observed are respectively 59 and 49% for the cell line Calu-3. An antiviral activity was also detected for the Vero E6 line, after treatment with TOTUM 4 to 6,667, at 10,000 ng/mL and at 20,000 ng/mL, for which viral loads of 78%, 66% and 40% were respectively observed.

It should also be noted that, in a particularly advantageous manner, during the assaying of the cytokines IL6 and IP10 which were carried out for CAS 1 and for CAS 2, a dose-response effect is observed for TOTUM 4, with decreasing concentrations of cytokines when the concentrations of compounds increase.

As a result of the above results, and in particular those relating to the assay of the IL6 and IP10 cytokines in lung cells, it is possible to assert that a diluted mother tincture extract, in particular mother tincture diluted to ¼, and freeze-dried, of leaves of Neurolaena lobata, in a liquid solution having a concentration of between 6,500 and 20,000 ng/mL (mass of freeze-dried mother tincture extract/volume of aqueous solution), preferably between 6,667 and 20,000 ng/mL, has an antiviral activity and a virucidal activity against the SARS-CoV-2 virus responsible for Covid-19, and exhibits efficacy in combating the severe forms of this disease.

Indeed, as the results demonstrate a dose-dependent dose effect of TOTUM 4 on the release of IL-6 and IP1O cytokines, such a mother tincture extract of Neurolaena lobata is particularly indicated in the optics to avoid the cytokine storm likely to occur, in particular, in the lungs of patients suffering from a severe form of Covid, in response to infection by the virus.

Here “patient suffering from a severe form of Covid-19” is understood to mean a patient hospitalized to combat Covid-19 and placed under oxygenotherapy.

1. A therapeutic composition comprising:
a mother tincture of a leaf extract of a plant of the genus “Neurolaena” and the species “lobata” for use as a drug inhibiting human dihydroorotate dehydrogenase (DHODH) in the treatment of a viral infection of RNA-genome virus,
wherein said RNA-genome virus is selected from viruses from the following list:
the viruses of the Coronaviridae family,
the virus responsible for hepatitis C, the virus responsible for yellow fever or the Zika virus in the Flaviviridae family,
the virus responsible for chikungunya in the Togaviridae family.

2. The therapeutic composition according to claim 1, wherein said RNA-genome virus is the SARS-CoV-2 virus responsible for Covid-19.

3. A method for preparing a dry extract of a diluted mother tincture of dried leaves of Neurolaena lobata, the method comprising the following steps:
i) preparing a mixture of dried leaves of Neurolaena lobata in sugar cane alcohol at 50°, said mixture having a mass concentration of between 15 and 20 g/L;
ii) macerating said mixture under agitation for about 21 days;
iii) filtering, after the step of macerating, the mixture on “cartridges” of porosity 50-75 μm and a liquid filtrate is obtained, corresponding to a mother tincture of dried leaves of Neurolaena lobata, and a solid retentate;
iv) diluting said liquid mother tincture to ¼ by adding an aqueous solution, and an aqueous-alcoholic solution is thus obtained;
v) evaporating the alcohol contained in this solution by a rotary evaporator, until an aqueous solution is obtained; and
vi) consolidating and freeze-drying the aqueous solution thus obtained to obtain a dry extract of the diluted mother tincture of dried leaves of Neurolaena lobata.

4. The method for preparing a dry extract from a diluted mother tincture of dried leaves of Neurolaena lobata according to claim 3, wherein the mixture prepared in step i) has a mass concentration comprised between 16 and 17 g of dried leaves of Neurolaena lobata per liter (L) of sugar cane alcohol at 50°, and preferably equal to 16 g/L.

5. The method for preparing a dry extract from a diluted mother tincture of dried leaves of Neurolaena lobata according to claim 3, wherein, during step iv), said mother tincture is diluted by mixing a volume equal to 0.75 L of filtrate and a volume equal to 2.25 L of water.

6. The method for preparing a liquid solution from a dry extract of freeze-dried diluted mother tincture of dried leaves of Neurolaena lobata, obtained, according to claim 3, wherein said liquid solution is obtained by diluting said dry extract in water or in a pharmaceutically acceptable aqueous solvent, said liquid solution having a concentration of between 6,500 and 20,000 ng of dry extract per mL of aqueous solvent, preferably between 6,667 and 20,000 ng/mL.

7. A diluted mother tincture extract of dried leaves of Neurolaena lobata, comprising: diluted to ¼ of the mother tincture, and freeze-dried, said extract being in a solution and having a final concentration of between 6,500 and 20,000 ng/mL (mass of freeze-dried dry extract of diluted mother tincture/volume of aqueous solution), for its use in the treatment of a viral infection due to the SARS-CoV-2 virus responsible for Covid-19.

8. The mother tincture extract of Neurolaena lobata leaves according to claim 7, wherein, for use as a medicament in the decrease in the production of cytokines, in particular IL-6 and IP-10, in the treatment of the serious forms of a viral infection due to the SARS-CoV-2 virus responsible for Covid 19 ame in the blowtorch (3).
